(19) United States
(12) Patent Application Publication
(10) Pub. No.: US 2006/0286046 A1
(43) Pub. Date: Dec. 21, 2006

(54) Skin care compositions

(76) Inventor: C. Andrew Haber, Toronto (CA)

Correspondence Address: GOWAN INTELLECTUAL PROPERTY, 1075 NORTH SERVICE ROAD WEST, SUITE 203, OAKVILLE, ON, L6M-2G2, CA

(21) Appl. No.: 11/028,616

(22) Filed: Jan. 5, 2005

(51) Int. Cl. A61K 8/49

(52) U.S. Cl. 424/59; 424/401; 514/554; 514/562; 514/548; 514/263.31

(57) ABSTRACT

A skin care composition is provided which preferably comprises a mixture of N-acetylcysteine and L-carnosine in combination with a cosmetic, dermatological or pharmaceutically acceptable carrier therefor. The composition may optionally be provided as a sunscreen formulation, and provides a method for the prevention, amelioration or treatment of pathological conditions of the skin, including, but not limited to, intrinsic or chronological aging, or aging due to sun damage (photoaging), and which conditions are caused by, or exacerbated by, oxidative stress, carbonyl stress, or a combination of both.

### FIELD OF THE INVENTION

The present invention relates to the field of cosmetic or dermatological skin care or treatment compositions, and in particular, relates to a specific mixture of a free radical scavenger and a reactive carbonyl scavenger, which are used in combination to prevent, ameliorate or treat pathological conditions of the skin, including but not limited to aging, which are caused, or exacerbated by oxidative stress, carbonyl stress, or a combination of both.

### BACKGROUND OF THE INVENTION

Human skin is a composite material of the epidermis and the dermis. The topmost part of the epidermis is the stratum corneum. This layer is the stiffest layer of the skin, as well as the one most affected by the surrounding environment. Below the stratum corneum is the internal portion of the epidermis. Below the epidermis, the topmost layer of the dermis is the papillary dermis, which is made of relatively loose connective tissues that define the micro-relief of the skin. The reticular dermis, disposed beneath the papillary dermis, is tight, connective tissue that is spatially organized. The reticular dermis is also associated with coarse wrinkles. At the bottom of the dermis lies the subcutaneous layer.

The principal functions of the skin include protection, excretion, secretion, absorption, thermoregulation, pigmentogenesis, accumulation, sensory perception, and regulation of immunological processes. These functions are detrimentally affected by the structural changes in the skin due to aging, disease, or exposure to solar radiation, pollution, and other factors present in the environment. The physiological changes associated with skin aging include impairment of the barrier function and decreased turnover of epidermal cells.

The mechanical properties of the skin, such as elasticity, are controlled by the density and geometry of the network of collagen and elastic fiber tissue therein. Damaged collagen and elastin lose their contractile properties, resulting in skin wrinkling and skin surface roughness. As the skin ages or becomes unhealthy, it acquires sags, stretch marks, bumps, bruises or wrinkles. Further, it roughens, and it has reduced ability to synthesize Vitamin D. Aged skin also becomes thinner and has a flattened dermoepidermal interface because of the alterations in collagen, elastin, and glycosaminoglycans.

Some pathological conditions of the skin such as intrinsic or chronological aging, sun damage (photodamage), are thought to be caused in large part by oxidative stress, which is an imbalance (at the cellular level), caused by decreased antioxidant capacity, increased production of reactive oxygen species (ROS) or both. Other factors such as cigarette smoking, and exposure to environmental contaminants such as ozone, are related to the development of undesirable changes in the skin due (in part) to oxidative stress. Reactive Oxidative Species (ROS) are readily formed or found on the skin, and they have been found to be damaging to the proteins, membranes and nucleic acids of the skin. For example, ROS are formed in response to common skin stresses, including exposure to solar radiation (especially UV radiation) and pollutants (extrinsic sources), and are a bi-product of normal and pathological cellular metabolism (intrinsic sources). Antioxidant systems exist to detoxify the cellular milleux, but these systems become less efficient with age, and can be overloaded when concentrations of ROS exceed their ability to cope. As an example, older individuals are more susceptible to solar radiation-induced acceleration of skin aging due to decreased antioxidant capacity relative to a younger individual. The outward signs of aging skin will also manifest in older individuals because a portion of sun damage to skin is irreversible and thus cumulative.

Skin epithelial cells, in particular, are a major target of oxidative stress.

To combat oxidative stress, anti-oxidants are commonly used in skin care compositions in order to assist in reducing the effects of the ROS. For example, vitamin E is used in skin care applications as an antioxidant, and the topical use of vitamin C is also believed to ward off sun damage, as well as reduce breakdown of connective tissues, and possibly promote collagen synthesis. Catechin-based preparations, including proanthanols and proanthocyanidins are also used and considered to be powerful antioxidants.

A wide variety of antioxidants are known and their use has been described in the prior art. For example, various antioxidants including cysteine or carnosine, amongst a host of other materials, are described in general terms in U.S. Pat. No. 6,800,293, with respect to the selection of an antioxidant for a typical skin care formulation.

Further, topical application of N-acetylcysteine had been proposed as a method for prevention of sunburn in EP 219455, for regulation of existing skin wrinkles and atrophy in U.S. Pat. No. 5,296,500, and for inhibition/prevention of photoaging, when combined with a sunblocking agent, to undamaged skin.

In addition to oxidative stress, increased carbonyl stress mediated by glycation can cause skin deterioration. Glycation is a process of spontaneous protein damage by reactive carbonyl compounds such as reducing sugars. In this scenario, cellular proteins are modified by reaction between reactive carbonyls and primary amino groups of proteins. A further chemical reaction known as the Maillard reaction leads to accumulation of what has been termed “Advanced Glycation End Products” (AGEs). The exact type of reactive carbonyl chemicals that are responsible for this process in human cells is not certain, but evidence suggests that carbohydrates such as glucose are primarily responsible.

Glycated skin proteins such as collagen, increase with age, and are enhanced in sun-damaged skin, and in certain diseases such as diabetes. AGEs cause damage to cellular proteins by several processes including cross linking of protein molecules, which decreases the solubility of the protein, and modifies its physical and metabolic properties. In addition, AGEs also mediate the damaging effects of solar radiation by absorbing UVA radiation, and generating free radicals via type 1-photoreaction, type 2-photoreaction or both.

Carnosine has been proposed as a nutritional supplement to accelerate wound healing in U.S. Pat. No. 5,656,588, and as a method for treatment of the complications and pathology of diabetes in U.S. Pat. No. 5,561,110. Sachdev in US Patent application publication No. 2004/0057974 proposes the use of carnosine and N-acetylcysteine, however, the preferred levels of carnosine are between 10 and 20% of the total formulation, and the optional levels of N-acetylcysteine described are less than 0.2%.

The prior art, however, does not teach cosmetic, dermatological, or pharmaceutical compositions or methods, for the prevention, amelioration or treatment of pathological conditions of the skin, including but not limited to intrinsic or chronological aging, and aging due to sun damage (photoaging), which are caused by, or exacerbated by, oxidative stress, carbonyl stress, or a combination of both, by using primarily a specific combination of a reactive carbonyl scavenger, and a free radical scavenger, and in particular the combination selected and described hereinbelow, with the intention of reinforcing with synergy, the activity of the later, with the former, and visa versa.

As such, while the prior art provides some beneficial effects, it would clearly be advantageous to provide a cosmetic, dermatological and/or pharmaceutical composition, and a method of use of the composition, which would provide additional utility in the prevention, amelioration and/or treatment of pathological conditions of the skin.

### SUMMARY OF THE INVENTION

Accordingly, it is a principal advantage of the present invention to provide a skin care composition that provides a method for the prevention, amelioration or treatment of pathological conditions of the skin, including but not limited to intinsic or chronological aging, and aging due to sun damage (photoaging), which conditions are caused by, or exacerbated by, oxidative stress, carbonyl stress, or a combination of both.

It is a further advantage of the present invention to provide such a composition in a suitable cosmetic, dermatological or pharmaceutically acceptable form.

The advantages set out hereinabove, as well as other objects and goals inherent thereto, are at least partially or fully provided by the skin care composition of the present invention, as set out herein below.

Accordingly, in one aspect, the present invention provides a skin care composition for the prevention, amelioration or treatment of pathological conditions of the skin, including but not limited to intrinsic or chronological aging, or aging due to sun damage (photoaging), which conditions are a result of, or exacerbated by, oxidative stress, carbonyl stress, or a combination of both, comprising an mixture of a free radical scavenger and a reactive carbonyl scavenger, and a cosmetic, dermatological or pharmaceutically acceptable carrier therefor.

In the practice of the present invention, suitable free radical scavengers are substances that either directly or indirectly protect cells against adverse effects of xenobiotics, drugs, carcinogens and toxic radical reactions. These include vitamin C (ascorbic acid), vitamin E (a-tocopherol), vitamin A, b-carotene, metallothionein, polyamines, melatonin, NADPH, adenosine, coenzyme Q-10, urate, ubiquinol, polyphenols, flavonoids, phytoestrogens, cysteine, homocysteine, taurine, methionine, s-adenosyl-L-methionine, resveratrol, nitroxides, GSH, glutathione peroxidase (GPX), superoxide dismutase (SOD), catalase (CAT), thioredoxin reductase, nitric oxide sintase (NOS), heme oxygenase-1 (HO-1), eosinophil peroxidase (EPO), or cosmetic, dermatological or pharmaceutically-acceptable salts and esters thereof.

However, a most preferred free radical scavenger is N-acetylcysteine.

Suitable reactive carbonyl scavengers include materials such as aminoguanidine, dimethylbiguanide (metformin), [(±)-5-[[4-[2-(5-ethyl-2-pyridinyl)ethoxy]phenyl]methyl]-2,4-]thiazolidine-dione (pioglitazone), 3,7-Dihydro-3,7dimethyl-1-(5-oxohexyl)-1H-purine-2,6-dione (pentoxyfylline), D-penicillamine, thiamine pyrophosphate, pyridoxamine, 2-[(2,6-Dichlorophenyl)amino]benzeneacetic acid (diclofenac), inositol, N-[(4-Amino-2-methyl-5-pyrimidinyl)methyl]-N-(4-hydroxy-2-mercapto-1-methyl-1-butenyl)formamide S-benzoate O-phosphate (benfotiamine), (±)-3-(2-thienyl)-2-piperazine (Tenilsetam), 3,4,5-trihydroxystilbene (resveratrol), (±)-2-isopropylidenhydrazono-4-oxo-thiazolidin-5-ylacetalinide (OPB-9195), diaminophenazine, LR-9,4-(2-naphtylcarboxamido)phenoxyisobutyric acid, LR-20, L-bis-4[-(4-chlorobenzamidophenoxyisobutyryl)cystine, LR-23, 4-(3,5-dichlorophenylureido-phenoxyisobutyryl-1-amidocyclohexane-1-carboxylic acid, LR-33, 4-(2-chloro-4-nitrophenylureido)phenoxyisobutyric acid, LR-41, 4-(3-chloro-4-fluorophenylureido)phenoxyisobutyric acid, LR-59, 4-[(3,4-dicholorophenylmethyl)2-chlorophenylureido]phenoxyisobutyric acid, LR-62, 4-(2,4-dichlorophenacylamino) phenoxyisobutyric acid, LR-74, 2-(8-quinolinoxy)propionic acid, LR-90, Methylene bis [4,4′-(2-chlorophenylureidophenoxyisobutyric acid)], LR-102, 1,4-benzene-bis[4-methyleneaminophenoxyisobutyric acid], LR-20, L-bis-4[-(4-chlorobenzamidophenoxyisobutyryl)cystine, LR-23, 4-(3,5-dichlorophenylureido)-phenoxyisobutyryl-1-amidocyclohexane-1-carboxylic acid, LR-99, 4-[(3,5-dichlorophenylureidophenoxyisobutyryl]-4-aminobenzoic acid)], LR-102, 1,4-benzene-bis [4-methyleneaminophenoxyisobutyric acid], SMR-5,5-aminosalicylic acid (5-ASA). Additionally, the cosmetic, dermatological or pharmaceutically-acceptable salts and esters of these agents can also be utilized.

A preferred reactive carbonyl scavenger is carnosine, and in particular, a most preferred reactive carbonyl scavenger is L-carnosine.

Consequently, in a most preferred embodiment of the present invention, the mixture of free radical and reactive carbonyl scavengers is a mixture of N-acetylcysteine and L-carnosine, respectively.

The relative ratio of free radical to reactive carbonyl scavengers in the skin care compositions of the present invention can vary widely depending on the degree of treatment required or desired, and the intended formulation and/or application. As such, the preferred relative ratio of free radical to reactive carbonyl scavengers ranges from 1:100 to 100:1. More preferably, the ratio of free radical to reactive carbonyl scavengers ranges from 1:5 to 5:1. Even more preferably, the ratio of free radical to reactive carbonyl scavenger ranges from 1:2 to 2:1.

The amount of free radical and reactive carbonyl scavengers present in the skin care composition can also vary widely depending on the treatment level required or desired, and the intended formulation and/or application. Preferably, however, the total level of free radical and reactive carbonyl scavengers present in the skin care composition is less than 40% by weight, more preferably less than 20% by weight, and even more preferably, less than 10%. Most preferably, however, the total level of free radical and reactive carbonyl scavengers present in the skin care composition is less than 5% by weight of the skin care composition.

Unless otherwise stated, all percentage values used herein, are provided on a weight basis.

As such, in a preferred embodiment, the present invention provides a skin care composition comprising: i) 0.1 to 20% by weight of a free radical scavenger, and preferably, N-acetylcysteine; ii) 0.25 to 20% of a reactive carbonyl scavenger, and preferably, L-carnosine; and, iii) a cosmetic, dermatological or pharmaceutically acceptable carrier therefore. More preferably, the level of free radical scavenger is between 0.2 and 5% by weight, still more preferably between 0.3 and 1%. Further, the level of reactive carbonyl scavenger is between 0.25 and 10%, still more preferably between 0.3 and 5%, and most preferably, between 1 and 2%.

In a most preferred embodiment, the present invention provides a skin care composition comprising i) 0.3 and 1% of N-acetylcysteine; ii) 1 to 2% of L-carnosine; and iii) a cosmetic, dermatological or pharmaceutically acceptable carrier therefore.

The preferred compositions are preferably manufactured so as to include L-carnosine and N-acetylcysteine which are added directly to the composition. However, in the practice of the present invention, compositions wherein these compounds are generated or released immediately before use, or are generated in-situ on the skin, also fall within the scope of the invention. This can include, for example, cosmetic, dermatological or pharmaceutically acceptable derivatives (salts, esters, ethers, sugars, nucleotides, nucleosides, peptides and lipids) of the active compounds which are also suitable according to the invention.

The carrier can include any of a number of cosmetic, dermatological or pharmaceutically acceptable carriers, as discussed in detail hereinbelow.

In a further aspect, the present invention also provides a composition as described hereinabove, which is used in the form of a sunscreen composition.

In a still further aspect, the present invention also provides a method for the prevention, amelioration or treatment of pathological conditions of the skin, including but not limited to aging or sun damage (photoaging), which conditions is as a result of, or exacerbated by, oxidative stress, carbonyl stress, or a combination of both, comprising applying to the skin, an mixture of a free radical scavenger and a reactive carbonyl scavenger, and a cosmetic, dermatological or pharmaceutically acceptable carrier therefor.

### DETAILED DESCRIPTION OF THE INVENTION

In the present application, the term “skin care composition” refers to any composition or material which is applied to the skin, in any number of different manners, in order to assist in the prevention, amelioration and/or treatment of pathological conditions of the skin including aging. Typically the pathological condition such as aging, will be a result, or exacerbated by, the effects of oxidative stress, carbonyl stress or a combination of both.

The process of free-radical- and carbonyl-damage of cellular components, such as sun damage of skin, as well as numerous dermatological conditions, are distinct but related processes that contribute to both chronological- and photo-damage of skin. Free radicals are known to increase reactive carbonyl species, which are involved in chemical reactions that lead to further production of free radicals. This cycle, termed a positive-feedback loop accelerates the production of both free radicals and reactive carbonyl species. As a result, oxidation and glycation of the components of a skin cell by these free radicals and reactive carbonyls respectively, can result in modification of the cellular components such as lipids, proteins and DNA.

By simultaneous use of both a free radical and a reactive carbonyl scavenger, it is believed that the production of any deleterious chemicals is reduced to a greater extent than the use of either material alone. As such, improved protection against these deleterious chemicals is provided.

N-acetylcysteine is the preferred free radical scavenger, and is a stabilized form of the amino acid cysteine. It is commonly obtained by purification from plant and animal sources. It was first used clinically as a mucolytic agent in the 1960s, and later to treat acetaminophen hepatotoxicity. It has since been used as an investigative tool in a wide range of research areas thought to involve free radicals, including cancer, cardiovascular diseases, human immunodeficiency virus (HIV) infections, metal toxicity, smoking, and diabetes. N-acetylcysteine is known to react directly with certain free radicals including hypochlorous acid, hydroxyl radical, and hydrogen peroxide. Also, by increasing levels of glutathione (GSH, a tri-peptide or chain of 3 amino acids), N-acetylcysteine increases the activity of glutathione peroxidase, an enzyme antioxidant that neutralizes free radicals such as hydrogen peroxide. Thus, N-acetylcysteine can work directly as a nonenzymatic antioxidant, and indirectly, as an antioxidant cofactor in the production of GSH.

It is also known that N-acetylcysteine is a precursor to glutathione (GSH), which is a general ROS scavenger. Direct treatment with GSH has limitations however, in that it does not penetrate well into cells within intact tissues. As such, the use of N-acetylcysteine provides improved performance in the compositions of the present invention.

It is known that L-carnosine acts as a carbonyl scavenger and anti-glycating agent. Accordingly, the preferred reactive carbonyl scavenger is L-carnosine since this material is associated with preventing carbonyl formation (especially in proteins) via random ROS-induced reactions.

The skin care composition of the present invention is preferably a cosmetic, cosmeceutical, pharmaceutical or dermatological preparation that is applied to the skin as a topical cream or liquid solution or dispersion. However, the physical form of the skin care composition is not critical. The compositions can also be, for example, formulated as bars, liquids, pastes, mousses, creams, gels, aerosols, lotions, hair shampoos, hair lotions, foam baths, shower baths, alcoholic and aqueous/alcoholic solutions, emulsions, wax/fat compounds, stick preparations, powders or ointments.

The skin care compositions of the present invention are preferably formulated to have a pH which is similar to the pH of the skin. As such, neutral or slightly acidic pH's are preferred. In particular, the skin care compositions of the present invention are formulated to have pH of between 4 and 7, and more preferably between 4.5 and 5.5.

The skin care compositions can be applied by spreading or wiping the composition on the skin, or by spraying, dusting, dipping or otherwise exposing the skin to the skin care composition.

Preferably, the skin is treated in a non-aerosol manner in order to avoid promotion of the evaporation of the components. Further, it is also preferred that the skin care compositions be stored and/or used in a fashion which minimizes contact with the air before application to the skin.

The “cosmetic, dermatological or pharmaceutically acceptable carrier”, as used herein, means one or more compatible solid or liquid filler diluents or microencapsulating substances which are suitable for administration to a human or lower animal. Preferred “carriers” must be of sufficiently high purity and sufficiently low toxicity to render them suitable for administration to the human or lower animal being treated. A safe and effective amount of carrier is from about 50% to about 99.5%, preferably from about 70% to about 99%, more preferably from about 80% to about 90%, of the composition.

Variations in formulation of these carriers will result in a wide variety of products which fall within the scope of the present invention.

The skin care compositions of the present invention may be made into a wide variety of product types. These include, but are not limited to lotions, creams, beach oils, gels, sticks, sprays, ointments, pastes, mousses and cosmetics. These product types may comprise several types of carrier systems including, but not limited to solutions, emulsions, gels and solids.

The skin care composition of the present invention formulated as solutions typically include a cosmetic, dermatological or pharmaceutically acceptable aqueous or organic solvent. The terms “solvent” refers to a material which is capable of having dispersed or dissolved therein the active compounds (namely, the free radical scavenger and the reactive carbonyl scavenger), and possesses acceptable safety properties (e.g., with respect to irritation and sensitization characteristics of the skin). Water is a typical aqueous solvent. Examples of suitable organic solvents include: propylene glycol, butylene glycol, polyethylene glycol (200-600), polypropylene glycol (425-2025), glycerol, 1,2,4-butanetriol, sorbitol esters, 1,2,6-hexanetriol, ethanol, isopropanol, butanediol, and mixtures thereof.

If the skin care compositions of the present invention are formulated as an aerosol and applied to the skin as a spray-on, a propellant is added to a solution composition. Examples of propellants useful herein include, but are not limited to, the chlorinated, fluorinated and chloro-fluorinated lower molecular weight hydrocarbons. A more complete disclosure of propellants useful herein can be found in Sagarin, Cosmetics Science and Technology, 2nd Edition, Vol. 2, pp. 443-465 (1972).

Skin care compositions of the present invention may be formulated as a solution comprising an emollient. An example of a composition formulated in this way would be a beach oil product. Preferably, such compositions contain from about 0.1% to about 10% of the active compounds and from about 2% to about 50% of a cosmetic, dermatological or pharmaceutically-acceptable emollient.

As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), incorporated herein by reference, contains numerous examples of suitable materials.

A lotion can be made from a solution carrier system. Lotions preferably comprise from about 0.1% to about 10%, more preferably from about 1% to about 5%, of the active compounds; from about 1% to about 20%, preferably from about 5% to about 10%, of an emollient; and from about 50% to about 90%, preferably from about 60% to about 80%, water.

Another type of product that may be formulated from a solution carrier system is a cream. A cream of the present invention would preferably comprise from about 0.1% to about 10%, more preferably from about 1% to about 5%, of the active compounds; from about 5% to about 50%, preferably from about 10% to about 20%, of an emollient, and from about 45% to about 85%, preferably from about 50% to about 75%, water.

Yet another type of product that may be formulated from a solution carrier system is an ointment. An ointment may comprise a simple base of animal or vegetable oils or semi-solid hydrocarbons (oleaginous). Ointments may also comprise absorption ointment bases which absorb water to form emulsions. Ointment carriers may also be water soluble. An ointment may also comprise from about 2% to about 10% of an emollient plus from about 0.1% to about 2% of a thickening agent. A more complete disclosure of thickening agents useful herein can be found in Segarin, Cosmetics, Science and Technology, 2nd Edition, Vol. 1, pp. 72-73 (1972).

If the carrier is formulated as an emulsion, from about 1% to about 10%, preferably from about 2% to about 5%, of the carrier system comprises an emulsifier. Emulsifiers may be nonionic, anionic or cationic. Suitable emulsifiers are disclosed in, for example, U.S. Pat. No. 3,755,560, issued Aug. 28, 1973, Dickert et al; U.S. Pat. No. 4,421,769, issued Dec. 20, 1983, Dixon et al.; and McCutcheon's Detergents and Emulsifiers, North American Edition, pages 317-324 (1986); the disclosures of which are incorporated herein by reference. Preferred emulsifiers are anionic or nonionic, although the other types may also be used.

Lotions and creams can be formulated as emulsions as well as solutions. Preferably such lotions comprise from about 0.1% to about 10%, more preferably from about 1% to about 5%, of the active compounds; from about 1% to about 20%, preferably from about 5% to about 10%, of an emollient; from about 25% to about 75%, preferably from about 45% to about 95%, water; and from about 0.1% to about 10%, preferably from about 0.5% to about 5%, of an emulsifier. Such creams would preferably comprise from about 0.1% to about 10%, more preferably from about 1% to about 5%, of the active compounds; from about 1% to about 20%, preferably from about 5% to about 10%, of an emollient; from about 20% to about 80%, preferably from about 30% to about 70%, water; and from about 1% to about 10%, preferably from about 2% to about 5%, of an emulsifier.

Single emulsion skin care preparations, such as lotions and creams, of the oil-in-water type and water-in-oil type are well-known in the cosmetic art and are useful in the present invention. Multiphase emulsion compositions, such as the water-in-oil-in-water type, as disclosed in U.S. Pat. No. 4,254,105, Fakuda et al., issued Mar. 3, 1981, incorporated herein by reference, are also useful in the present invention. In general, such single or multiphase emulsions contain water, emollients and emulsifiers as essential ingredients.

Triple emulsion carrier systems comprising an oil-in-water-in-silicone fluid emulsion composition as disclosed in U.S. Pat. No. 4,960,764, Figueroa, issued Oct. 2, 1990, are also useful in the present invention. Preferably, this triple emulsion carrier system can be combined with from about 0.1% to about 10%, more preferably from about 1% to about 5%, of the active compounds to yield the skin care compositions of the present invention.

Another emulsion carrier system useful in the skin care compositions of the present invention is a microemulsion carrier system. An example of such a system comprises from about 9% to about 15% squalane; from about 25% to about 40% silicone oil; from about 8% to about 20% of a fatty alcohol; from about 15% to about 30% of polyoxyethylene sorbitan monofatty acid (commercially available under the trade name Tweens) or other nonionics; and from about 7% to about 20% water. This carrier system is preferably combined with from about 1% to about 5% of the active compounds.

If the skin care compositions of the present invention are formulated as a gel or a cosmetic stick, a suitable amount of a thickening agent, as disclosed herein, is added to a cream or lotion formulation.

The skin care compositions of the present invention may also be formulated as makeup products such as foundations.

The skin care compositions of the present invention may contain, in addition to the aforementioned components, a wide variety of additional oil-soluble materials and/or water-soluble materials conventionally used in topical compositions, at their art-established levels.

Various water-soluble materials may also be present in the compositions of this invention. These include humectants, proteins and polypeptides, preservatives and an alkaline agent. In addition, the topical compositions herein can contain conventional cosmetic adjuvants, such as dyes, opacifiers (e.g., titanium dioxide), pigments and perfumes.

The skin care compositions of the present invention may also include a safe and effective amount of a penetration enhancing agent. A preferred amount of penetration enhancing agent is from about 1% to about 5% of the composition. Examples of useful penetration enhancers, among others, are disclosed in U.S. Pat. No. 4,537,776, Cooper, issued Aug. 27, 1985; U.S. Pat. No. 4,552,872, Cooper et al., issued Nov. 12, 1985; U.S. Pat. No. 4,557,934, Cooper, issued Dec. 10, 1985; U.S. Pat. No. 4,130,667, Smith, issued Dec. 19, 1978; U.S. Pat. No. 3,989,816, Rhaadhyaksha, issued Nov. 2, 1976; U.S. Pat. No. 4,017,641, DiGiulio, issued Apr. 12, 1977; and U.S. Pat. No. 4,954,487, Cooper et al., issued Sep. 4, 1990.

Other conventional skin care product additives may also be included in the compositions of the present invention. For example, collagen, hyaluronic acid, elastin, hydrolysates, primrose oil, jojoba oil, epidermal growth factor, soybean saponins, mucopolysaccharides, and mixtures thereof may be used.

Various vitamins may also be included in the compositions of the present invention. For example, vitamin A, and derivatives thereof, vitamin B₂, biotin, pantothenic, vitamin D, vitamin E, and mixtures thereof may be used.

The skin care compositions of the present invention may also comprise, in addition to the active compounds, a cosmetically-acceptable surfactant. The term “cosmetically-acceptable surfactant” refers to a surfactant or emulsifier which is not only an effective skin cleanser, but also can be used without undue toxicity, irritation, allergic response, and the like. Furthermore, the surfactant or emulsifier must be capable of being commingled with the active compounds in a manner such that there is no interaction which would substantially reduce the efficacy of the composition for regulating skin wrinkles and/or skin atrophy.

The skin care compositions of the present invention may contain from about 0.1% to about 20%, preferably from about 1% to about 5%, of the active compounds and from about 1% to about 90%, more preferably from about 5% to about 10%, of a cosmetically-acceptable surfactant.

The surfactant component of the compositions of the present invention are selected from anionic, nonionic, zwitterionic, amphoteric and ampholytic surfactants, as well as mixtures of these surfactants. Such surfactants are well-known to those skilled in the detergency art.

Typical examples of suitable mild, i.e. particularly cosmetic, dermatologically or pharmaceutically acceptable emulisifiers or surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, .alpha.-olefin sulfonates, ether carboxylic acids, alkyl oligoglucosides, fatty acid glucamides, alkylamidobetaines and/or protein fatty acid condensates, preferably based on wheat proteins.

When formulated as “cleaning compositions”, the skin care composition of the present invention can optionally contain, at their art-established levels, any additional suitable known materials which are conventionally used in skin cleansing compositions.

While the compositions described hereinabove might be used in any suitable format, as described hereinabove, the skin care compositions of the present invention might additional comprise any of a number of additional materials described hereinabove, or otherwise designed to provide acceptable application properties, or provide additional cosmetic, pharmaceutical or dermatological effects.

These additional materials can be, for example, superfatting agents, pearlizing waxes, consistency factors, thickeners, polymer additives, silicone compounds or derivatives, fats, oils, waxes, stabilizers, biogenic agents, deodorizers, anti-dandruff agents, film formers, foam stabilizers, electrolytes, swelling agents, UV protection factors, hydrotropes, preservatives, bactericides, perfumes and/or perfume oils, antifoams, dyes, pigments which have a coloring effect, moisturizers and/or humectants, insect repellents, self-tanning agents, solubilizers, germ inhibitors, anti-inflammatory agents, benofuran derivatives, retinoids, chelating agents, and the like as further auxiliaries and additives.

Some of these additional materials are discussed in detail hereinbelow.

However, it should also be noted that an additional content of other antioxidants, in addition to the free radical and/or reactive carbonyl scavenger antioxidants identified hereinabove, may also be included in the compositions of the present invention.

Antioxidants have been defined as substances “that when present at low concentrations compared with those of an oxidizable substrate (i.e. a free radical target molecule), significantly delay or prevent oxidation of that substrate”. Generally, antioxidants fall into one of four categories, namely: 1) antioxidant enzymes, 2) metal-binding proteins (preventative antioxidants), 3) non-enzymatic antioxidants, and 4) antioxidant co-factors. Antioxidants that are found in prior art cosmetic products are almost exclusively of the non-enzymatic type, and include: vitamins A, B (e.g. nicotinamide), C, and E, lipoic acid, carotenoids (e.g. beta-carotene, lycopene, etc.), ubiquinone (coenzyme Q10), and plant extracts (the antioxidant capacity of plant extracts is usually ascribed to a class of molecules known as phenols with more than 8,000 phenolic structures currently known).

The additional antioxidants which might also be used in the skin care compositions, and preferably, in combination with a combination of N-acetylcysteine and L-carnosine, are all preferably antioxidants which are considered suitable for skin care composition applications. These additional antioxidants are preferably selected from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, methyl, ethyl, propyl, amyl, butyl and lauryl, palmitoyl, oleyl, linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to 11 mol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

The amount of the abovementioned additional antioxidants (one or more compounds) in the preparations is preferably from 0.001 to 30% by weight, more preferably from 0.05 to 20% by weight, and most preferably 1 to 10% by weight, based on the total weight of the composition.

Suitable oil components which might be use are, for example, Guerbet alcohols based on fatty alcohols containing 6 to 18 and preferably 8 to 10 carbon atoms, esters of linear C₆₋₂₂ fatty acids with linear C₆₋₂₂ fatty alcohols, esters of branched C₆₋₁₃ carboxylic acids with linear C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of hydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-/di-/triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, Guerbet carbonates, esters of benzoic acid with linear and/or branched C₆₋₂₂ alcohols (for example Finsolv™. TN), linear or branched, symmetrical or nonsymmetrical dialkyl ethers containing 6 to 22 carbon atoms per alkyl group, ring opening products of epoxidized fatty acid esters with polyols, silicone oils and/or aliphatic or naphthenic hydrocarbons, for example squalane, squalene or dialkyl cyclohexanes.

The superfatting agents used may be such substances as, for example, lanolin and lecithin and polyethoxylated or acylated lanolin and lecithin derivatives, polyol fatty acid esters, monoglycerides and fatty acid alkanolamides, the latter also serving as foam stabilizers.

Suitable pearlizing waxes are, for example, alkylene glycol esters, particularly ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, for example fatty alcohols, fatty ketones, fatty aldehydes, fatty ethers and fatty carbonates which contain a total of at least 24 carbon atoms, especially laurone and distearyl ether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups; and mixtures thereof.

Suitable secondary consistency factors are hydroxyfatty alcohols, partial glycerides, fatty acids or hydroxyfatty acids. Suitable thickeners are, for example, Aerosil types (hydrophilic silicas), polysaccharides, more particularly xanthan gum, guar guar, agar agar, alginates and tyloses, carboxymethyl cellulose and hydroxyethyl cellulose, relatively high molecular weight polyethylene glycol monoesters and diesters of fatty acids, polyacrylates (for example Carbopols™ or Synthalens™), polyacrylamides, polyvinyl alcohol and polyvinyl pyrrolidone, surfactants such as, for example, ethoxylated fatty acid glycerides, esters of fatty acids with polyols such as, for example, pentaerythritol or trimethylol propane, narrow-range fatty alcohol ethoxylates or alkyl oligoglucosides and electrolytes, such as sodium chloride and ammonium chloride.

Suitable cationic polymers are, for example, cationic cellulose derivatives such as, for example, the quaternized hydroxyethyl cellulose obtainable from Amerchol under the name of Polymer JR 400™, cationic starch, copolymers of diallyl ammonium salts and acrylamides, quaternized vinyl pyrrolidone/vinyl imidazole polymers such as, for example, Luviquat™, condensation products of polyglycols and amines, quaternized collagen polypeptides such as, for example, Lauryidimonium Hydroxypropyl Hydrolyzed Collagen (Lamequat™), quaternized wheat polypeptides, polyethyleneimine, cationic silicone polymers such as, for example, Amodimethicone, copolymers of adipic acid and dimethylamino-hydroxypropyl diethylenetriamine (Cartaretine™), copolymers of acrylic acid with dimethyl diallyl ammonium chloride (Merquat™ 550), polyaminopolyamides, and crosslinked water-soluble polymers thereof, cationic chitin derivatives such as, for example, quaternized chitosan, optionally in micro-crystalline distribution, condensation products of dihaloalkyls, for example dibromobutane, with bis-dialkylamines, for example bis-dimethylamino-1,3-propane, cationic guar gum such as, for example, Jaguar™ CBS, Jaguar™ C-17, Jaguar™ C-16, quaternized ammonium salt polymers such as, for example, Mirapol™ A-15, Mirapol™ AD-1, Mirapol™ AZ-1.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

Typical examples of fats are glycerides while suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes, microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes.

Metal salts of fatty acids such as, for example, magnesium, aluminium and/or zinc stearate or ricinoleate may be used as stabilizers.

Biogenic agents might also be used, and these include, for example, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes.

Suitable deodorizers are, for example, antiperspirants, such as aluminium chlorhydrates. These antiperspirants are preferably colorless hygroscopic crystals which readily deliquesce in air and which accumulate when aqueous aluminium chloride solutions are concentrated by evaporation. An aluminium chlorhydrate for use in the compositions of the present invention is commercially available under the name of Locron™. Besides the chlorhydrates, aluminium hydroxylactates and acidic aluminium/zirconium salts may also be used. Other suitable deodorizers are esterase inhibitors, preferably trialkyl citrates, such as trimethyl citrate, tripropyl citrate, triisopropyl citrate, tributyl citrate and, in particular, triethyl citrate (Hydagen™ CAT). Esterase inhibitors inhibit enzyme activity and thus reduce odor formation. The free acid is probably released through the cleavage of the citric acid ester, reducing the pH value of the skin to such an extent that the enzymes are inhibited. Other esterase inhibitors are sterol sulfates or phosphates, for example lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester.

Antibacterial agents which influence the germ flora and destroy or inhibit the growth of perspiration-decomposing bacteria, may also be present. Examples of such antibacterial agents are chitosan, phenoxyethanol and chlorhexidine gluconate. 5-Chloro-2-(2,4-dichlorophenoxy)-phenol, which is marketed under the name of Irgasan™, may also be used.

Pathological conditions leading to oxidative stress are commonly associated with inflammation. Therefore, it is preferable that an anti-inflammatory agent be included as an active agent along with the composition.

A safe and effective amount of an anti-inflammatory agent may therefore be added to the composition of the present invention, at levels of preferably from about 0.1% to about 10%, and more preferably from about 0.5% to about 5% of the composition. The exact amount of anti-inflammatory agent to be used in the compositions will depend on the particular anti-inflammatory agency utilized due to the wide variation in potency of such agents.

Steroidal anti-inflammatory agents, including but not limited to, corticosteroids such as hydrocortisone, hydroxyltriamcinolone, alpha-methyl dexamethasone, dexamethasone-phosphate, beclomethasone dipropionate, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, difluprednate, flucloronide, flunisolide, fluoromethalone, fluperolone, fluprednisolone, hydrocortisone valerate, hydrocortisone cyclopentylpropionate, hydrocortamate, meprednisone, paramethasone, preunisolone, preunisone, beclomethasone dipropionate, triamcinolone, and mixtures thereof may be used. The preferred steroidal anti-inflammatory for use in the present invention is hydrocortisone.

A second class of anti-inflammatory agents which is useful in the compositions of the present invention includes the non-steroidal anti-inflammatory agents. Specific non-steroidal anti-inflammatory agents useful in the composition of the present invention include, but are not limited to: the oxicams, salicylates, acetic acid derivatives, fenamates, propionic acid derivatives, and pyrazoles.

Mixtures of these non-steroidal anti-inflammatory agents may also be employed, as well as the pharmaceutically-acceptable salts and esters of these agents. For example, etofenamate, a flufenamic acid derivative, is particularly useful for topical application. Of the nonsteroidal anti-inflammatory agents, ibuprofen, naproxen, flufenamic acid, mefenamic acid, meclofenamic acid, piroxicam and felbinac are preferred; ibuprofen, naproxen, and flufenamic acid are most preferred.

Another class of anti-inflammatory agents which are useful in the present invention are the anti-inflammatory agents disclosed in U.S. Pat. No. 4,708,966, Loomans et al., issued Nov. 24, 1987. This patent discloses a class of nonsteroidal anti-inflammatory compounds which comprise specifically substituted phenyl compounds, especially substituted 2,6-di-tert-butyl phenol derivatives. For example, compounds selected from 4-(4′-pentyn-3′-one)-2,6-di-t-butylphenol; 4-(5′-hexynoyl)-2,6-di-t-butylphenol; 4-((S)-(31)-3′-methyl-5′-hexynoyl)-2,6-di-t-butylphenol; 4-((R)-(+)-3′-methyl-5′-hexynoyl)-2,6-di-t-butylphenol; and 4-(3′,3′-dimethoxypropionyl)-2,6-di-t-butylphenol are useful in the present invention.

Yet another class of anti-inflammatory agents which are useful in the present invention are those disclosed in U.S. Pat. No. 4,912,248, Mueller, issued Mar. 27, 1990. This patent discloses compounds and diastereomeric mixtures of specific 2-naphthyl-containing ester compounds, especially naproxen ester and naproxol ester compounds, having two or more chiral centers. For example, compounds selected from (S)-naproxen-(S)-2-butyl ester, (S)-naproxen-(R)-2-butylester, (S)-naproxol-(R)-2-methyl butyrate, (S)-naproxol-(S)-2-methyl butyrate, diasteromeric mixtures of (S)-naproxen-(S)-2-butyl ester and (S)-naproxen-(R)-2-butyl ester, and diasteromeric mixtures of (S)-naproxol-(R)-2-methyl butyrate and (S)-naproxol-(S)-2-methyl butyrate are useful in the present invention.

Finally, naturally derived, or so-called “natural” anti-inflammatory agents are useful in the present invention. For example, candelilla wax, alpha bisabolol, aloe vera, Manjistha (extracted from plants in the genus Rubia, particularly Rubia Cordifolia), and Guggal (extracted from plants in the genus Commiphora, particularly Commiphora Mukul), may be used.

Another preferred composition of the present invention comprises the active compound, a sunscreen, and an anti-inflammatory agent together for skin protection in the amounts disclosed for each individually herein.

In a preferred composition of the present invention, a benzofuran derivative, preferably amiodarone, is included as an active agent along with the active compound. The inclusion of a benzofuran derivative can increase the protective benefits of the composition.

A safe and effective amount of a benzofuran derivative may be added to the compositions of the present invention, preferably from about 0.01% to about 20%, more preferably from about 0.1% to about 10%, of the composition. Benzofuran derivatives useful in the present invention are disclosed in U.S. Pat. No. 5,118,707, Chatterjee and Kapoor, issued Jun. 2, 1992, incorporated herein by reference.

The inclusion of a retinoid can increase the protective benefits of the composition. Accordingly, in a preferred embodiment, the skin care compositions of the present invention comprises a retinoid, and in particular, retinoic acid. A safe and effective amount of a retinoid may be added to the compositions of the present invention, preferably from about 0.001% to about 2%, more preferably from about 0.01% to about 1% of the composition. As used herein, “retinoid” includes all natural and/or synthetic analogs of Vitamin A or retinal-like compounds which possess the biological activity of Vitamin A in the skin as well as the geometric isomers and stereoisomers of these compounds, such as all-trans retinoic acid and 13-cis-retinoic acid.

In a preferred composition of the present invention, a chelating agent is included as an active agent along with the active compound. As used herein, “chelating agent” means an active agent capable of removing a metal ion from a system by forming a complex so that the metal ion cannot readily participate in or catalyze chemical reactions. The inclusion of a chelating agent increases the benefits of the protective composition.

Chelating agents in protective barrier creams have often been used in the prevention of allergic contact dermatitis to metals, and to prevent the transition metal chemistry which can lead to the production of free radicals. A safe and effective amount of a chelating agent may be added to the compositions of the present invention, preferably from about 0.1% to about 10%, more preferably from about 1% to about 5%, of the composition. Chelators useful in compositions of the present invention are disclosed in U.S. Pat. No. 5,487,884 issued to Bissett, Bush & Chatterjee, and incorporated herein by reference. Preferred chelators useful in compositions of the present invention are ethylenediaminepentaacetic acid (EDTA) and diethylenetriaminepenta-acetic acid (DTPA).

In a preferred composition of the present invention, compositions comprise one, any two, any three, any four, or all five of a sunscreening agent, anti-inflammatory agent, benzofuran derivative, retinoid, and/or chelating agent, in addition to the free radical and reactive carbonyl scavenger. The inclusion of two, three, four, or all five of these agents with the active compound increases the protective benefits of the composition.

It will be clear that one possible form of the skin care composition of the present invention is as a sunscreen. In this application, the sunscreen will preferably additionally comprise at least one UV-A filter and/or at least one UV-B filter and/or at least one inorganic pigment, preferably an inorganic micropigment.

Examples of UV protection factors which might be used include organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances: 3-benzylidene camphor or 3-benzylidene norcamphor and derivatives thereof, for example 3-(4-methylbenzylidene)-camphor; 4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester; esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene); esters of salicylic acid, preferably salicylic acid-2-ethylhexyl ester, salicylic acid-4-isopropylbenzyl ester, salicylic acid homomenthyl ester; derivatives of benzophenone, preferably 2-hydroxy-4-methoxybenzo-phenone, 2-hydroxy-4-methoxy-4′-methylbenzophenone, 2,2′-dihydroxy-4-methoxybenzophenone; esters of benzalmalonic acid, preferably 4-methoxybenzalmalonic acid di-2-ethylhexyl ester; triazine derivatives such as, for example, 2,4,6-trianilino-(p-carbo-2′-ethyl-1′-hexyloxy)-1,3,5-triazine and Octyl Triazone; propane-1,3-diones such as, for example, 1-(4-tert.butylphenyl)-3-(4′-methoxyphenyl)-propane-1,3-dione; ketotricyclo(5.2.1)decane derivatives.

Suitable water-soluble substances are 2-phenylbenzimidazole-5-sulfonic acid and alkali metal, alkaline earth metal, ammonium, alkylammonium, alkanolammonium and glucam-monium salts thereof; sulfonic acid derivatives of benzophenones, preferably 2-hydroxy-4-methoxybenzophenone-5-sulfonic acid and salts thereof; sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example 1-(4′-tert.butylphenyl)-3-(4′-methoxyphenyl)-propane-1,3-dione, 4-tert-butyl-4′-methoxydibenzoylmethane (Parsol 1789) or 1-phenyl-3-(4′-isopropylphenyl)-propane-1,3-dione. The UV-A and UV-B filters may of course also be used in the form of mixtures.

Besides the soluble substances mentioned, insoluble pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have an average diameter of less than 100 nm, preferably from 5 to 50 nm and more preferably from 15 to 30 nm. They may be spherical in shape although ellipsoidal particles or other non-spherical particles may also be used. The pigments may also be surface-treated, i.e. hydrophilicized or hydrophobicized. Typical examples are coated titanium dioxides such as, for example, Titandioxid T 805 or Eusolex™ T2000. Suitable hydrophobic coating materials are, above all, silicones and especially trialkoxyoctyl silanes or simethicones. So-called micro- or nanopigments are preferably used in sun protection products. Micronized zinc oxide is preferably used.

In addition, hydrotropes such as, for example, ethanol, isopropyl alcohol or polyols may be used to improve flow behavior. Suitable polyols preferably contain 2 to 15 carbon atoms and at least two hydroxyl groups. The polyols may contain other functional groups, especially amino groups, or may be modified with nitrogen. Typical examples are glycerol; alkylene glycols such as, for example, ethylene glycol, diethylene glycol, propylene glycol, butylene glycol, hexylene glycol and polyethylene glycols having an average molecular weight of 100 to 1,000 dalton; technical oligoglycerol mixtures with a degree of self-condensation of 1.5 to 10 such as, for example, technical diglycerol mixtures with a diglycerol content of 40 to 50% by weight; methylol compounds such as, in particular, trimethylol ethane, trimethylol propane, trimethylol butane, pentaerythritol and dipentaerythritol; lower alkyl glucosides, particularly those containing 1 to 8 carbon atoms in the alkyl group, for example methyl and butyl glucoside; sugar alcohols containing 5 to 12 carbon atoms such as, for example, sorbitol or mannitol; sugars containing 5 to 12 carbon atoms such as, for example, glucose or sucrose; aminosugars such as, for example, glucamine; dialcoholamines, such as diethanolamine or 2-aminopropane-1,3-diol.

Suitable preservatives are, for example, phenoxyethanol, formaldehyde solution, parabens, pentanediol or sorbic acid.

A suitable insect repellents are N,N-diethyl-m-toluamide, pentane-1,2-diol.

A suitable self-tanning agent is dihydroxyacetone.

Suitable perfume, or other masking agents, including perfume oils, are mixtures of natural and synthetic fragrances. Natural fragrances include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamon, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, .alpha.-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams.

It is preferred to use mixtures of different perfume compounds which, together, produce an agreeable fragrance.

Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, labolanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evemyl, iraldein gamma, phenylacetic acid, geranyl acetate, benzyl acetate, rose oxide, romillat, irotyl and floramat.

Suitable dyes are any of the substances suitable and approved for cosmetic purposes. These dyes are normally used in concentrations of 0.001 to 0.1% by weight, based on the skin care composition as a whole.

Typical examples of germ inhibitors are preservatives which act specifically against gram-positive bacteria such as, for example, 2,4,4′-trichloro-2′-hydroxydiphenyl ether, chlorhexidine (1,6-di-(4-chlorophenyl-biguanido)-hexane) or TCC (3,4,4′-trichlorocarbanilide). Numerous perfumes and essential oils also have antimicrobial properties. Typical examples are the active substances eugenol, menthol and thymol in clove, mint and thyme oil. An interesting natural deodorant is the terpene alcohol farnesol (3,7,11-trimethyl-2,6,10-dodecatrien-1-ol) which is present in linden blossom oil and which smells of lily-of-the-valley. Glycerol monolaurate has also been successfully used as a bacteriostatic agent. The percentage content of the additional germ-inhibiting agents is normally about 0.1 to 2% by weight, based on the solids component of the preparations.

It will be clear, however, that a wide variety of formulations and mixtures can be used depending on the intended format of the skin care composition to be used in the practice of the present invention. Those skilled in the art will be readily able to prepare suitable compositions which include any of the above named additional materials. The total percentage content of such auxiliaries and additives may preferably be from 1 to 50% by weight and more preferably, is from 5 to 40% by weight, based on the particular composition. The preparations may be produced by standard techniques known to those skilled in the art.

### BRIEF DESCRIPTION OF THE DRAWINGS

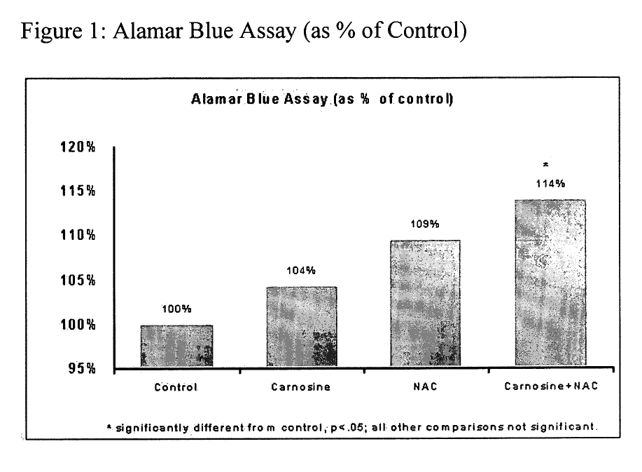

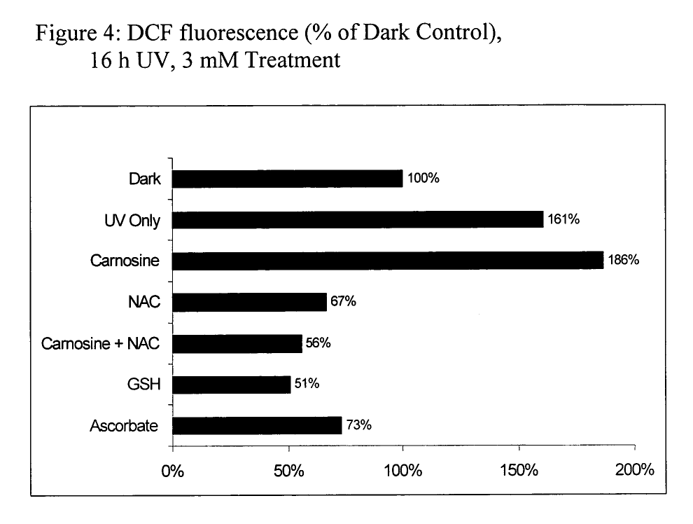

The results of a series of experiments, as discussed hereinbelow, are shown on the enclosed drawings, wherein FIGS. 1 and 4 are charts showing the performance results of the experiments described herein.

### EXAMPLES

The novel features which are believed to be characteristic of the present invention, as to its structure, organization, use and/or method of operation, together with further objectives and advantages thereof, will be better understood from the following examples in which a presently preferred embodiment of the invention will be discussed, by way of example only. It is expressly understood, however, that the examples are for the purpose of illustration and description only and are not intended as a definition of the limits of the invention.

The experiments compare the protective effects of various antioxidants using in vitro models of oxidative stress. The results contained herein demonstrate that the combination of N-acetylcysteine (NAC) and carnosine confers superior protection when compared to other common antioxidants such as vitamin C (ascorbate) and GSH. Furthermore, the experiments are supportive of a synergistic effect of NAC and carnosine.

In the following set of experiments, the effect on metabolic activity, and the efficiency of preventing ROS induced biological damage, of various materials on JEG-3 cells was studied under conditions that are known to induce oxidative stress. JEG-3 cells are human epithelial carcinoma cells derived from reproductive tissue. As epithelial cells, they are a good surrogate for surface cells (including skin cells).

Metabolic Activity

Alamar Blue™ is a metabolic activity indicator that requires the reducing power of the cell to transform the dye from the non-fluorescent to the fluorescent. Decreased metabolic activity of a cell is generally associated with cell damage due to a variety of stressors.

JEG-3 cells, a human placental choriocarcinoma cell line of epithelial origin, were grown and maintained at 37° C., 5% CO₂ in 75 mm² culture flasks until a confluent monolayer was formed. Cells were then removed, washed, re-suspended, and counted on a hemocytometer, allowing for the concentration to be adjusted to 30,000 cells/mL. Aliquots of 0.2 mL of the cell solution were added into each well of a 96 well micro-plate. The cells were incubated at 37° C., 5% CO₂ for 48 hours prior to treatment to allow for a confluent monolayer to form. The media was aspirated off and new media with test compounds were added at various concentrations.

Dark Exposure

All treatments were made in Minimal Essential Media (MEM) without Fetal Bovine Serum (FBS). The anti-oxidants were added directly to the medium. Dark exposures involved exposing the cells to a combination of control (no chemicals added), or antoxidants. After the addition of the chemicals, the cells were incubated in the dark for 24 hours at 37° C./5% CO₂.

Simulated Solar Radiation (SSR) Exposure

All treatments were made in MEM without FBS. The anti-oxidants were added directly to the medium. The cells were then incubated for 16 hours at 37° C./5% CO₂ under simulated solar radiation (SSR) conditions, with a UVB:UVA:visible light ratio of 0.3:5.52:96 mmol m−2 s−1 (total fluence rate: 102 mmol m−2 s−1, which is about 5% of full sunlight).

Viability Experiments

Two viability experiments were done at the end of the incubation periods (Dark or SSR exposure). Alamar Blue™ is a metabolic (primarily mitochondrial) activity indicator that requires the reducing power of the cell to transform the dye from non-fluorescent to fluorescent. Carboxy-fluorescein diacetate (CFDA-AM) is also a fluorescent dye that requires cellular esterases inside a cell to become fluorescent. The presence of an intact membrane allows the CFDA-AM to concentrate and give a higher reading than may occur outside the cell. Both dyes were combined in phosphate buffered saline with glucose (PBSG) buffer and added to the microplate wells after the media (MEM plus treatment) was removed. The indicators were used at concentrations suggested by the manufacturer. The plates were incubated for 30 minutes at 37° C./5% CO₂ and were then read on a fluorimeter (PerSeptive Biosystems Cytofluor 4000) with excitation and emission, respectively, set at 530 nm and 590 nm for Alamar Blue™ and 485 nm and 530 nm for CFDA-AM. Because of the different excitation and emission wavelengths as well as little cross-reactivity, both dyes can be used simultaneously. The results were calculated as a percentage of the control value.

Reactive Oxygen Species Detection

To determine the levels of ROS generated in the cell system, another fluorescent probe, H2DCFDA, was used. After the 16-hour incubation period under SSR, the media was removed. The probe was added to PBSG in a ratio of 1 μl/ml for a final concentration of 4 nM according to manufacturer instructions. From this mixture, 200 μl/well was added and the plate read on the Cytofluor 4000 at 485 nm excitation and 530 nm emission for 4 hours with readings taken every hour. Results are stated in relative fluorescence units representing the fluorescent product, dichlorofluorescein (DCF). The results were calculated as a percentage of the dark control.

Results

1. Dark Experiments

JEG-3 cells incubated in the dark for 24 hours with both N-acetylcysteine(NAC) and carnosine, showed a modest improvement in metabolic activity compared to control cells, while use of NAC or carnosine alone did not differ significantly from control (FIG. 1). This supports the possibility of a synergistic protective effect of the combination of the free radical scavenger (NAC) and the carbonyl scavenger (carnosine).

2. SSR Experiments

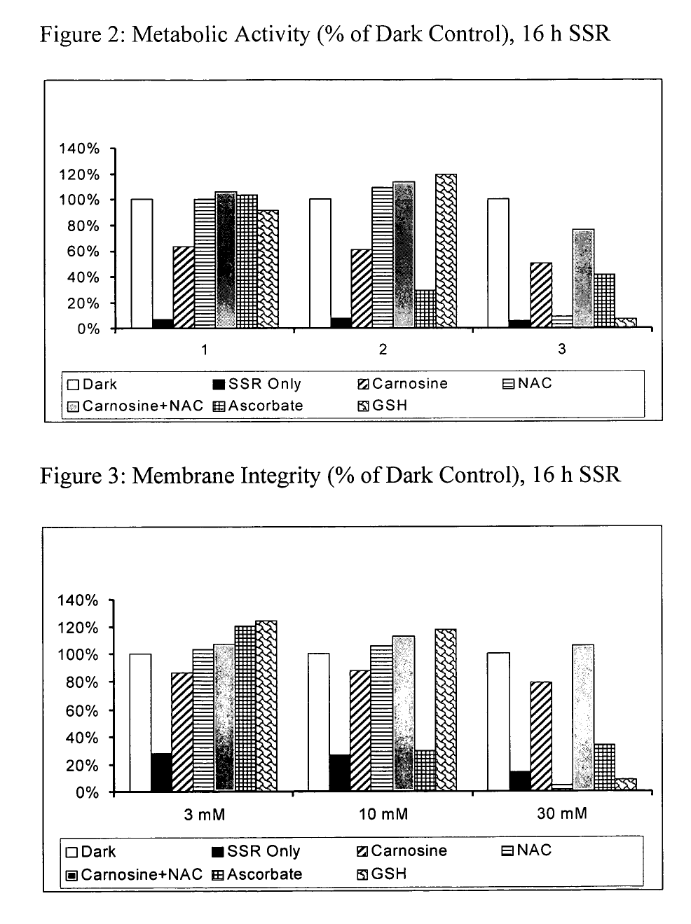

Viability of JEG-3 cells exposed to SSR for 16 hours was drastically reduced as determined by Alamar Blue and CFDA assay. Treatment with antioxidants showed varying degrees of protection depending on the concentrations employed (FIG. 2 and FIG. 3). Taken together, NAC+carnosine offered superior protection to all other antioxidants used over the range of concentrations employed. Significantly, at 10 mM and 30 mM concentrations the protective effect of NAC+carnosine was far superior to ascorbate, traditionally considered one of the most powerful antioxidants. There was a trend for the antioxidant effect of NAC to be improved by combination with carnosine, supporting the possibility of a synergistic effect of the two compounds.

At 3 mM concentration (which equates to approximately 0.05% by weight of NAC and 0.07% of Carnosine), all antioxidants except carnosine prevented the decrease in cell viability caused by SSR, and were significantly different from the SSR exposed cells. As expected treatment with carnosine at all concentrations only provided for partial protection of cell viability because the cellular effect of carnosine is thought to be primarily related to it's carbonyl scavenging ability, whereas SSR damage is mediated primarily by a free radical mechanism.

At 10 mM concentration, NAC, NAC+carnosine, and GSH provided complete protection of cell viability whereas ascorbate and carnosine provided only partial protection. All effects were significantly different from SSR-exposed cells except for ascorbate.

At 30 mM concentration, carnosine+NAC provided the best protection of cell viability which was near complete as measured by CFDA assay and ˜75% as measured by Alamar Blue assay. All other antioxidants provided varying degrees of partial protection. Only the effects of carnosine and NAC+Carnsosine were significantly different from SSR-exposed cells.

Results from the ROS-indicator dye (H2DCF-DA) assay showed an increase in ROS levels with UV treatment with respect to a dark control (FIG. 4). As expected treatment with Carnosine did not decrease ROS levels because the cellular effect of Carnosine is thought to be primarily related to it's carbonyl scavenging ability. Treatment with NAC decreased dye levels (DCF production) indicating a decrease in intracellular ROS. The protective effect of NAC was superior to ascorbate but not as great as GSH. There was a trend for the antioxidant effect of NAC to be improved by combination with Carnosine, supporting the possibility of a synergistic effect of the two compounds.

As a result of these experiments, it can be seen that L-carnosine and N-acetylcysteine are more effective as a mixture than as individual compounds, and that the improved effect is synergistic. This is shown effectively in metabolic activity test and the ROS detection experiments.

Thus, it is apparent that there has been provided, in accordance with the present invention, a skin care composition which fully satisfies the goals, objects, and advantages set forth hereinbefore. Therefore, having described specific embodiments of the present invention, it will be understood that alternatives, modifications and variations thereof may be suggested to those skilled in the art, and that it is intended that the present specification embrace all such alternatives, modifications and variations as fall within the scope of the appended claims.

Additionally, for clarity and unless otherwise stated, the word “comprise” and variations of the word such as “comprising” and “comprises”, when used in the description and claims of the present specification, is not intended to exclude other additives, components, integers or steps.

Moreover, the words “substantially” or “essentially”, when used with an adjective or adverb is intended to enhance the scope of the particular characteristic; e.g., substantially planar is intended to mean planar, nearly planar and/or exhibiting characteristics associated with a planar element.

Further, use of the terms “he”, “him”, or “his”, is not intended to be specifically directed to persons of the masculine gender, and could easily be read as “she”, “her”, or “hers”, respectively.

Also, while this discussion has addressed prior art known to the inventor, it is not an admission that all art discussed is citable against the present application.

We claim:

1. A skin care composition for the prevention, amelioration or treatment of pathological conditions of the skin, which composition comprises a mixture of a free radical scavenger and a reactive carbonyl scavenger, and a cosmetic, dermatological or pharmaceutically acceptable carrier therefor.

2. A skin care composition as claimed in claim 1 wherein said pathological condition is caused by intrinsic or chronological aging or aging due to sun damage.

3. A skin care composition as claimed in claim 1 wherein said pathological condition is as a result of, or exacerbated by, the effects of oxidative stress, carbonyl stress or a combination of both.

4. A skin care composition as claimed in claim 1 wherein said free radical scavengers are substances that either directly or indirectly protect cells against adverse effects of xenobiotics, drugs, carcinogens and toxic radical reactions.

5. A skin care composition as claimed in claim 4 wherein said free radical scavenger is vitamin C (ascorbic acid), vitamin E (a-tocopherol), vitamin A, b-carotene, metallothionein, polyamines, melatonin, NADPH, adenosine, coenzyme Q-10, urate, ubiquinol, polyphenols, flavonoids, phytoestrogens, cysteine, homocysteine, taurine, methionine, s-adenosyl-L-methionine, resveratrol, nitroxides, GSH, glutathione peroxidase (GPX), superoxide dismutase (SOD), catalase (CAT), thioredoxin reductase, nitric oxide sintase (NOS), heme oxygenase-1 (HO-1), eosinophil peroxidase (EPO), or cosmetic, dermatological or pharmaceutically-acceptable salts and esters thereof.

6. A skin care composition as claimed in claim 1 wherein said free radical scavenger is N-acetylcysteine.

7. A skin care composition as claimed in claim 1 wherein said reactive carbonyl scavenger is aminoguanidine, dimethylbiguanide (metformin), [(±)-5-[[4-[2-(5-ethyl-2-pyridinyl)ethoxy]phenyl]methyl]-2,4-]thiazolidine-dione (pioglitazone), 3,7-Dihydro-3,7dimethyl-1-(5-oxohexyl)-1H-purine-2,6-dione (pentoxyfylline), D-penicillamine, thiamine pyrophosphate, pyridoxamine, 2-[(2,6-Dichlorophenyl)amino]benzeneacetic acid (diclofenac), inositol, N-[(4-Amino-2-methyl-5-pyrimidinyl)methyl]-N-(4-hydroxy-2-mercapto-1-methyl-1-butenyl)formamide S-benzoate O-phosphate (benfotiamine), (+)-3-(2-thienyl)-2-piperazine (Tenilsetam), 3,4,5-trihydroxystilbene (resveratrol), (±)-2-isopropylidenhydrazono-4-oxo-thiazolidin-5-ylacetalinide (OPB-9195), diaminophenazine, LR-9,4-(2-naphtylcarboxamido)phenoxyisobutyric acid, LR-20, L-bis-4[-(4-chlorobenzamidophenoxyisobutyryl)cystine, LR-23, 4-(3,5-dichlorophenylureido-phenoxyisobutyryl-1-amidocyclohexane-1-carboxylic acid, LR-33, 4-(2-chloro-4-nitrophenylureido)phenoxyisobutyric acid, LR-41, 4-(3-chloro-4-fluorophenylureido)phenoxyisobutyric acid, LR-59, 4-[(3,4-dicholorophenylmethyl)2-chlorophenylureido]phenoxyisobutyric acid, LR-62, 4-(2,4-dichlorophenacylamino) phenoxyisobutyric acid, LR-74, 2-(8-quinolinoxy)propionic acid, LR-90, Methylene bis [4,4′-(2-chlorophenylureidophenoxyisobutyric acid)], LR-102, 1,4-benzene-bis[4-methyleneaminophenoxyisobutyric acid], LR-20, L-bis-4[-(4-chlorobenzamidophenoxyisobutyryl)cystine, LR-23, 4-(3,5-dichlorophenylureido)-phenoxyisobutyryl-1-amidocyclohexane-1-carboxylic acid, LR-99, 4-[(3,5-dichlorophenylureidophenoxyisobutyryl]-4-aminobenzoic acid)], LR-102, 1,4-benzene-bis [4-methyleneaminophenoxyisobutyric acid], SMR-5,5-aminosalicylic acid (5-ASA), or cosmetic, dermatological, or pharmaceutically-acceptable salts and esters thereof.

8. A skin care composition as claimed in claim 1 wherein said reactive carbonyl scavenger is carnosine.

9. A skin care composition as claimed in claim 1 wherein said reactive carbonyl scavenger is L-carnosine.

10. A skin care composition as claimed in claim 1 wherein said free radical scavenger and said reactive carbonyl scavenger are N-acetylcysteine and L-carnosine, respectively, or a cosmetic, dermatological or pharmaceutically acceptable derivatives thereof.

11. A skin care composition as claimed in claim 1 wherein the relative ratio of free radical to reactive carbonyl scavengers is in the range of from 1:100 to 100:1.

12. A skin care composition as claimed in claim 1 wherein the relative ratio of free radical to reactive carbonyl scavengers is in the range of from 1:5 to 5:1.

13. A skin care composition as claimed in claim 1 comprising up to 40% by weight of a mixture of a free radical scavenger and a reactive carbonyl scavenger, and a cosmetic, dermatological or pharmaceutically acceptable carrier therefor.

14. A skin care composition as claimed in claim 1 comprising up to 20% by weight of a mixture of a free radical scavenger and a reactive carbonyl scavenger, and a cosmetic, dermatological or pharmaceutically acceptable carrier therefor.

15. A skin care composition as claimed in claim 1 comprising: i) 0.1 to 20% by weight of a free radical scavenger; ii) 0.25 to 20% of a reactive carbonyl scavenger; and, iii) a cosmetic, dermatological or pharmaceutically acceptable carrier therefore.

16. A skin care composition as claimed in claim 1 comprising: i) 0.1 to 20% by weight of N-acetylcysteine; ii) 0.25 to 20% of L-carnosine; and, iii) a cosmetic, dermatological or pharmaceutically acceptable carrier therefore.

17. A skin care composition as claimed in claim 16 wherein the level of N-acetylcysteine is between 0.2 and 5%.

18. A skin care composition as claimed in claim 17 wherein the level of N-acetylcysteine is between 0.3 and 1%.

19. A skin care composition as claimed in claim 16 wherein the level of L-carnosine is between 0.25 and 10%.

20. A skin care composition as claimed in claim 19 wherein the level of L-carnosine is between 0.3 and 5%.

21. A skin care composition as claimed in claim 1 comprising i) 0.3 to 1% of N-acetylcysteine; ii) 1 to 2% of L-carnosine; and iii) a cosmetic, dermatological or pharmaceutically acceptable carrier therefore.

22. A skin care composition as claimed in claim 1 additionally comprising further auxiliaries or additives selected from the group consisting of surfactants, superfatting agents, pearlizing waxes, consistency factors, thickeners, polymers, organic solvents, silicone compounds or derivatives, fats, oils, waxes, stabilizers, biogenic agents, deodorizers, anti-dandruff agents, film formers, swelling agents, UV protection factors, hydrotropes, preservatives, bactericides, perfumes, antifoams, dyes, pigments which have a coloring effect, thickeners, moisturizers and/or humectants, insect repellents, self-tanning agents, solubilizers, perfume oils, dyes, germ inhibitors.

23. A skin care composition as claimed in claim 22 wherein the total percentage content of such auxiliaries and additives is within the range of from 1 to 50% by weight based on the total weight of said composition.

24. A skin care composition as claimed in claim 1 additionally comprising cosmetic auxiliaries selected from the group consisting of alcohols, polyols, polymers, foam stabilizers, and electrolytes.

25. A skin care composition as claimed in claim 1 comprising additional antioxidants.

26. A skin care composition as claimed in claim 25 wherein said additional antioxidants are selected from the group consisting of amino acids and derivatives thereof, imidazoles and derivatives thereof, peptides and derivatives thereof, carotenoids, carotenes and derivatives thereof, chlorogenic acid and derivatives thereof, lipoic acid and derivatives thereof, aurothioglucose, propylthiouracil and other thiols and salts thereof, dilauryl thiodipropionate, distearyl thiodipropionate, thiodipropionic acid and derivatives thereof and sulfoximine compounds, chelating agents, α-hydroxy acids, humic acid, bile acid, bile extracts, bilirubin, biliverdin, EDTA, EGTA and derivatives thereof, unsaturated fatty acids and derivatives thereof, folic acid and derivatives thereof, ubiquinone and ubiquinol and derivatives thereof, vitamin C and derivatives, tocopherols and derivatives, vitamin A and derivatives, coniferyl benzoate of benzoin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof, selenium and derivatives thereof, stilbenes and derivatives thereof, and their salts, esters, ethers, sugars, nucleotides, nucleosides, peptides and lipids thereof.

27. A skin care composition as claimed in claim 26 wherein the amount of said additional antioxidants, in the composition, is between 0.001 to 30% by weight, based on the total weight of said composition.

28. A skin care composition as claimed in claim 1 wherein said skin care composition is formulated as a sunscreen composition.

29. A sunscreen composition comprising i) N-acetylcysteine; ii) L-carnosine, by weight; iii) a UV protection factor; and iv) a cosmetic, dermatological or pharmaceutically acceptable carrier therefore.

30. A sunscreen composition as claimed in claim 29 wherein said UV protection factor comprises at least one UV-A filter, at least one UV-B filter, or a finely dispersed metal oxides or salt thereof.

31. A method for the prevention, amelioration or treatment of pathological conditions of the skin comprising applying to the skin, an mixture of a free radical scavenger and a reactive carbonyl scavenger, and a cosmetic, dermatological or pharmaceutically acceptable carrier therefor.

32. A method as claimed in claim 31 wherein said pathological conditions are caused by intrinsic or chronological aging, or aging due to sun damage.

33. A method as claimed in claim 31 said pathological conditions are as a result of, or exacerbated by, the effects of oxidative stress, carbonyl stress or a combination of both.

34. A method as claimed in claim 31 wherein said free radical scavenger is N-acetylcysteine.

35. A method as claimed in claim 31 wherein said reactive carbonyl scavenger is L-carnosine
